(19) United States
(12) Patent Application Publication
(10) Pub. No.: US 2015/0011873 A1
(43) Pub. Date: Jan. 8, 2015

(54) CATHETERS, CATHETERS FOR USE IN ULTRASOUND GUIDED PROCEDURES, AND RELATED METHODS

(71) Applicant: N. Sandor Racz, Farmers Branch, TX (US)

(72) Inventor: N. Sandor Racz, Farmers Branch, TX (US)

(73) Assignee: Custom Medical Applications, Inc., Johnstown, NY (US)

(21) Appl. No.: 14/379,249

(22) PCT Filed: Feb. 16, 2012
(86) PCT No.: PCT/US12/25489

§ 371 Date: Aug. 15, 2014

(51) Int. Cl. A61B 8/08; A61M 25/00; A61B 10/00; A61M 25/01

(52) U.S. Cl. CPC A61B 8/0841; A61M 25/0108; A61M 25/007; A61B 10/0045; A61B 2010/0077
USPC 600/424; 604/528; 600/581; 29/868

(57) ABSTRACT

Catheters include a catheter tube and an echogenic structure extending from a proximal portion of the catheter tube to a distal end of the catheter tube where a portion of the echogenic structure is at least partially formed in the distal end of the catheter tube. Such catheters may exhibit improved echogenicity. Methods of forming a tool for a clinical procedure performed upon a subject utilizing ultrasound guidance include forming a catheter and forming a portion of an echogenic structure within a distal end of the catheter. Methods of ultrasound guidance for a clinical procedure performed upon a subject may include such catheters.

### CROSS-REFERENCE TO RELATED APPLICATION

This application is a national phase entry under 35 U.S.C. §371 of International Patent Application PCT/US2012/025489, filed Feb. 16, 2012, designating the United States of America and published in English as International Patent Publication WO 2013/122592 A1 on Aug. 22, 2013.

### TECHNICAL FIELD

The disclosure relates generally to the field of medical devices and related methods. In particular, the disclosure relates to catheters for use proximate portions of the nervous system of a subject, catheters having improved echogenic properties, methods for utilizing ultrasound to guide such catheters, and methods of forming such catheters.

### BACKGROUND

As described in U.S. Pat. No. 6,019,724 to Gronningsaeter et al. (Feb. 1, 2000), the disclosure of which is hereby incorporated herein in its entirety by this reference, ultrasound can provide useful feedback to a clinician during a clinical procedure.

With the advent of ever more sophisticated ultrasound equipment (e.g., a SONOSITE® NANOMAXX®, S SERIES®, MICROMAXX®, or M-TURBO® device, details available on the internet at sonosite.com) having ever improved resolution, the ability of a clinician to optimally place and use a tool, such as a cutting or resecting device, coagulating device, stapler, biopsy forceps, needle, cannula, etc., increases.

For example, improper placement of a catheter for delivering anesthesia can result in 20% to 40% secondary block failure. See, e.g., Tui & Bhargava, Atlas of Ultrasound and Nerve Stimulation-Guided Regional Anesthesia, 16.1 (2007).

Unfortunately, however, with the utilization of tools in the tightest crevices of the body near vital organs and tissues, comes the added risk associated with guiding and positioning the tools within the body and the added risk associated with unexpected tool failure.

### DISCLOSURE

Described are catheters that may be utilized with ultrasound guidance and exhibit improved echogenic properties, methods of utilizing such catheters, and methods of forming such catheters. Such catheters may include one or more structures therein that increase the echogenicity of the catheter (e.g., for improved ultrasound guidance). In some embodiments, the catheters may be used to transport fluid (e.g., a medication) to locations proximate (e.g., adjacent) one or more portions of the nervous system of a subject (e.g., proximate the spinal cord or peripheral nervous system of a subject). For example, the catheter may be placed adjacent to neural structures of the peripheral nervous system of a subject to form a nerve block.

In some embodiments, disclosed is a catheter including a catheter tube comprising a proximal end, a distal end, and a lumen formed therebetween. An echogenic structure extends from a proximal portion of the catheter tube to a distal portion of the catheter tube, a distal end of the echogenic structure being at least partially formed in the catheter tube proximate the distal end of the catheter tube.

In some embodiments, the echogenic structure may comprise an enlarged distal end having a diameter that is greater than a cross-sectional diameter of the echogenic structure and the enlarged distal end may be at least partially formed in the catheter tube proximate the distal end of the catheter tube.

In some embodiments, the distal end of the catheter tube may comprise a solid distal end.

In additional embodiments, disclosed is a catheter having improved echogenicity including a catheter tube having an open proximal end in communication with a lumen and a distal end. An echogenic structure extends through the lumen from the proximal end to proximate the distal end of the catheter tube. The echogenic structure terminates in a weld bead at least partially embedded within a distal portion of the catheter tube proximate the distal end of the catheter tube. The echogenic structure and the weld bead may provide improved imaging by an ultrasound sensor used to guide placement of the catheter and means for removing broken portions of the catheter during use.

In yet additional embodiments, disclosed is a catheter comprising a catheter tube including a proximal end, a distal end, and a lumen formed therebetween, the catheter tube having a plurality of apertures formed in a sidewall of the catheter tube at a distal portion of the catheter tube enabling fluid transported by the catheter to pass therethrough. The catheter further includes at least two discrete sets of wires where each set comprises a twisted pair of wires extending from a proximal portion of the catheter tube to a distal portion of the catheter tube. The at least two discrete sets of wires comprise an enlarged terminal end having a diameter that is greater than a cross-sectional diameter of at least one wire of the at least two discrete sets of wires where the enlarged terminal end is at least partially formed in the catheter tube proximate the distal end of the catheter tube. A remaining portion of each set of the at least two discrete sets of wires extends through the catheter tube separate from at least another set of the at least two discrete sets of wires.

In yet additional embodiments, disclosed is a method of fanning a tool for a clinical procedure performed upon a portion of a peripheral nervous system of a subject utilizing ultrasound guidance comprising forming a catheter having a lumen extending from a proximal end of the catheter to a solid distal end of the catheter, disposing an echogenic structure within the lumen of the catheter extending from a proximal portion of the catheter to a distal portion of the catheter, and forming an enlarged end of the echogenic structure within the catheter proximate the solid distal end of the catheter.

Also disclosed are methods of utilizing a catheter according to the disclosure.

### BRIEF DESCRIPTION OF THE DRAWINGS

FIG. 1 depicts an embodiment of the disclosure associated with a catheter connector hub.

FIG. 2 depicts the embodiment of the preceding figure with a portion of a catheter connector hub shown in cross-section.

FIG. 3 depicts an enlarged, partial cross-sectional view of a distal portion of the catheter of the embodiment of the preceding two figures.

FIG. 4 depicts an enlarged, partial cross-sectional view of a distal portion of a catheter in accordance with another embodiment of the disclosure.

FIG. 5 depicts an enlarged, partial cross-sectional view of a distal portion of a catheter in accordance with yet another embodiment of the disclosure.

FIG. 6 depicts an enlarged, partial cross-sectional view of a distal portion of a catheter in accordance with yet another embodiment of the disclosure.

FIG. 7 depicts an enlarged, partial cross-sectional view of a distal portion of a catheter in accordance with yet another embodiment of the disclosure.

FIG. 8 depicts an enlarged, partial cross-sectional view of a distal portion of a catheter in accordance with yet another embodiment of the disclosure.

FIG. 9 depicts an enlarged, partial cross-sectional view of a distal portion of a catheter in accordance with yet another embodiment of the disclosure.

FIG. 10 is a cross-sectional view of an embodiment of a catheter inserted within a subject.

### DETAILED DESCRIPTION

Illustrations presented herein are not meant to be actual views of any particular catheter or clinical procedure tool, but are merely idealized representations, which are employed to describe embodiments of the disclosure. Additionally, elements common between figures may retain the same numerical designation.

As described in U.S. Pat. No. 5,490,845 to Racz (Feb. 13, 1996), the disclosure of which is hereby incorporated herein in its entirety by this reference, “Small diameter catheters are used to introduce medication into the spinal canal, spinal space, epidural space, blood vessels, body cavities and the like. Due to their uni-wall construction when undergoing repetitive movement while being subjected to body heat, such small diameter catheters have a tendency to migrate to other body cavities or to kink thereby preventing the flow of medication therethrough. Such problems can be particularly troublesome when a catheter is used within the spinal canal. In the event of migration of the catheter any kinking of the catheter will preclude aspiration and seeing evidence of such migration due to the closure of the lumen of the catheter and the attendant inability to withdraw blood or spinal fluid. Typical prior art catheter placement units for small diameter catheters are shown in U.S. Pat. Nos. 3,856,009; 4,518,383; 4,650,472; 5,084,022; 5,106,376; 5,129,889; 5,213,578; and 5,232,442 (the disclosure of each of which is hereby incorporated herein in its entirety by this reference). Another problem associated with the use of such small diameter catheters is their susceptibility to breaking and, possibly, leaving portions thereof remaining in a body cavity. Removal of such broken portions of the catheter may be difficult or impossible.”

These problems were addressed in U.S. Pat. No. 5,490,845 by preventing occlusion of the catheter to allow the flow of fluids therethrough and allowing the removal of broken portions thereof from the spinal canal, spinal space, epidural space, blood vessels, body cavities, and the like during use. Specifically, as described by U.S. Pat. No. 5,899,891 to Racz (May 4, 1999), the disclosure of which is hereby incorporated herein in its entirety by this reference, U.S. Pat. No. 5,490,845 disclosed a flexible catheter that includes a catheter tube containing an intraluminal cord member (cord) extending along the tube's length and protruding out of the tube's distal and proximal ends. The thus placed cord helps to prevent collapse of the tube during fluid administration, and the portion of the cord extending out of the tube's distal end also aids in the retention and removal of parts of the tube, which might break off during use of the catheter.

The incorporated U.S. Pat. No. 5,899,891 goes on to describe “catheters which utilize a flexible tube that has been modified at either one end or both ends. Such modifications generally involve strengthening the interior of a tube end by increasing its break strength and possibly its rigidity, but do not generally involve decreasing the tip's flexibility. In one embodiment, the tube end modifications involve special placement of an intraluminal cord contained within the tube.”

The disclosure includes catheters and related methods wherein the catheter tube has been modified in such a way as to be echogenic and to enhance the ability to create an echo (i.e., return a signal in an ultrasound procedure). The catheters may include one or more structures that increase the echogenicity of the catheter placed at least partially within the tube. In some embodiments, the echogenic structure placed within the catheter tube may also act to support and reinforce portions of the catheter tube in such a manner that the reinforced portion of the catheter tube remains flexible. In some embodiments, the echogenic structure of the catheter may include a cord (e.g., a wire, a ribbon, or combinations thereof) running therethrough. The cord may be attached at the catheter or to additional echogenic structures such as, for example, an enlarged end (e.g., a ball or spherical member).

In some embodiments, the echogenic structure may extend into the distal portion of the catheter (e.g., proximate to the distal tip of the catheter). Such placement may enhance ultrasound imaging and may improve structural integrity of the catheter. In some embodiments, the echogenic structure may enable fluid (e.g., a medication such as, for example, an anesthetic, an analgesic, or combinations thereof) to pass therethrough in order to fluidly couple the lumen of the catheter tube with openings formed in the catheter tube (e.g., at the sidewalls or distal end thereof).

In use, the echogenic structure acts to increase the echogenicity of the catheter for better detection by ultrasound, to increase the rigidity of the catheter (while maintaining flexibility). Where the echogenic structure is implemented as at least one of a cord and ball structure, the echogenic structure may be utilized to collect a portion of the tube that has broken off in a procedure.

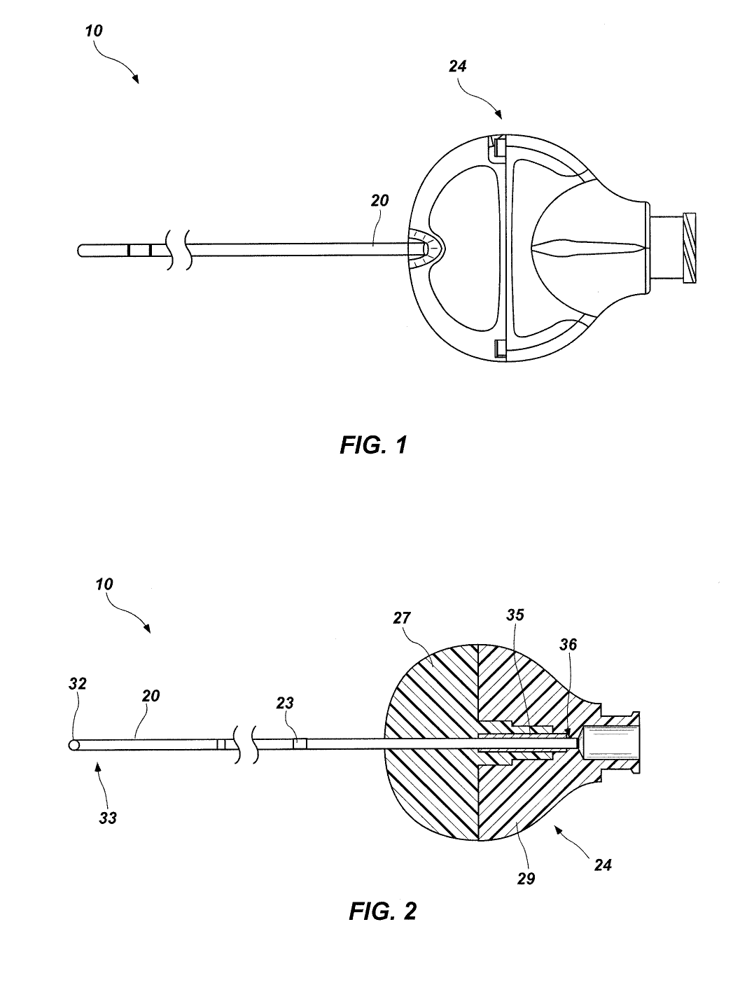

As shown in FIGS. 1 and 2, a catheter 10 may be used in conjunction with, for example, a catheter connector hub 24. In some embodiments, the catheter connection hub 24 may be similar to, for example, the catheter connection hubs described in U.S. Pat. No. 8,038,667, issued Oct. 18, 2011, the disclosure of which is hereby incorporated herein in its entirety by this reference. As shown, the catheter 10 includes a hollow cylindrical member such as a catheter tube 20. In some embodiments, the catheter tube 20 may, for example, have a length from about twenty-five (25) centimeters (9.8 inches) to about ninety (90) centimeters (about 35.4 inches) (e.g., sixty-one (61) centimeters (about 24 inches)). In some embodiments, the catheter tube 20 may, for example, have a diameter of about 0.762 millimeter (about 0.03 inch).

Referring to FIG. 2, the connector hub 24 is shown in conjunction with the catheter tube 20. The proximal portion 36 of the catheter tube 20 is securable within a catheter-receiving portion 27 of the catheter hub 24 using, for example, a deformable member 35 that may selectively constrict and expand in response to relative rotation of first and second portions 27 and 29 of the catheter hub 24.

In some embodiments, the catheter 10 may include a plurality of depth indicators 23 located on the catheter tube 20. The depth indicators 23 are configured to indicate to a user the depth of insertion of a distal end 32 of the catheter 10, and are typically located at positions corresponding to depths of insertion for certain procedures (e.g., depths relative to a flexible introducer cannula (FIC)).

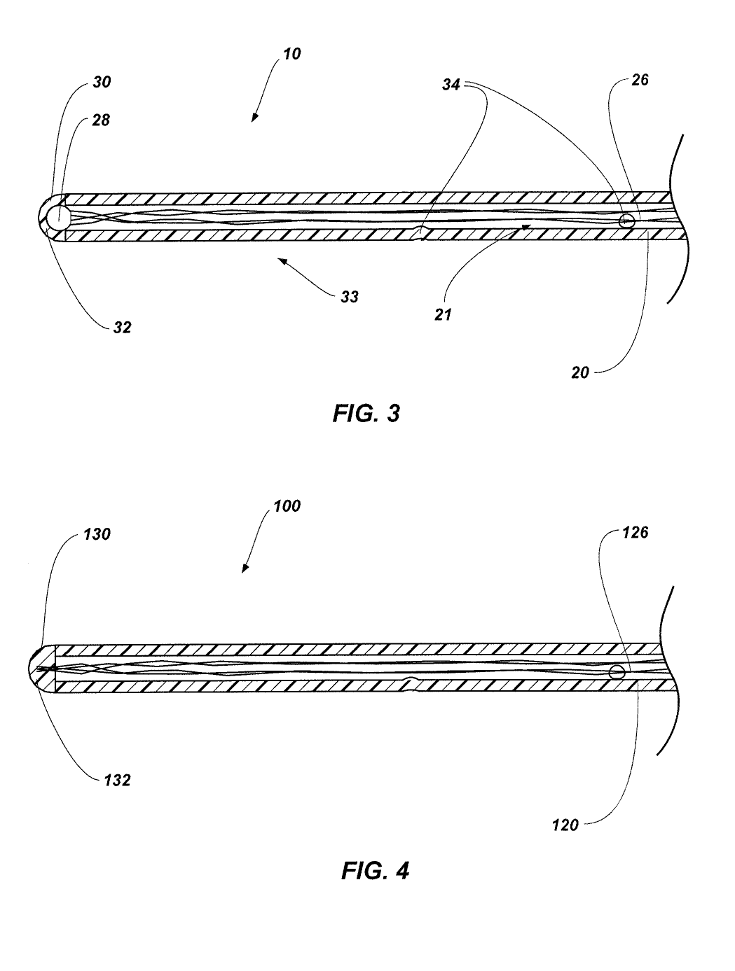

FIG. 3 depicts an enlarged, partial cross-sectional view of the distal portion 33 of the catheter 10 shown in FIGS. 1 and 2. Referring to FIG. 3, an echogenic structure may be placed at least partially within the catheter tube 20. For example, one or more wires 26 may be placed at least partially within the catheter tube 20. The wires 26 may extend through the catheter tube 20 and terminate proximate to the distal end 32 of the catheter tube 20 (e.g., in the distal portion 33). In some embodiments, the wires 26 may be one or more sets of twisted wires (e.g., one or more twisted pairs) forming a cord. For example, the wires 26 may include two or more sets (e.g., sets of twisted pairs). The sets of wires 26 may be configured to extend through the lumen 21 of the catheter 10 at least partially spaced from one another. For example, the sets of wires 26 may be configured to be spaced along a portion (e.g., a majority) of the catheter 10. Stated in another way, the sets of wires 26 may be substantially discrete except for, for example, being connected (e.g., directly or indirectly) at the distal end of the wires 26 to a common structure (e.g., to the catheter tube 20 or an enlarged end as discussed below) and/or at a proximal end of the wires 26 to another common structure (e.g., to a portion of the catheter 10 or to a catheter connection hub 24 (FIG. 2)). The discrete sets of wires 26 may extend separately (e.g., unattached) through a middle portion of the lumen 21. In other words, one set of wires 26 may not be connected with another set of wires 26 in the lumen 21 at locations between the distal and proximal ends of the wires 26. Such spacing of the sets of wires 26 in lumen 21 may enhance the echogenicity of the catheter 10. For example, as shown in FIG. 3, the spaced sets of wires 26 may occupy relatively more portions of a cross-sectional area of the lumen 21 as compared to a single wire or a tightly wound set of wires, thereby, enhancing the ability of the lumen 21 to be identified in an ultrasound procedure.

The wires 26 may extend through the lumen 21 of the catheter tube 20, and be free to move within the lumen 21 of the catheter tube 20. Referring also back to FIG. 2, in embodiments where the wires 26 are not somehow affixed to the proximal portion 36 of the catheter tube 20, the wires 26 may be secured in the catheter connector hub 24.

The wires 26 for use with catheters such as those disclosed herein may include a cable having several wound metal wires (e.g., stainless steel wires). The wires 26 can also be a braided line including at least three optionally braided cables. Such cords are strong, hypoallergenic, and flexible. In other embodiments, the wires 26 may be made of a single metal wire, other electrically conductive material, plastic (e.g., nylon), other polymers, silk, or other suitable material. In some embodiments, the wires 26 have a diameter of about 0.23 millimeter (about 0.009 inch). In some embodiments, the wires 26 may be manufactured to contain anti-thrombogenic agents or other materials, so as to prevent, for example, occlusion of the catheter during long-term use. In additional embodiments, other chemical agents may be introduced such as, for example, antiseptics and anesthetics.

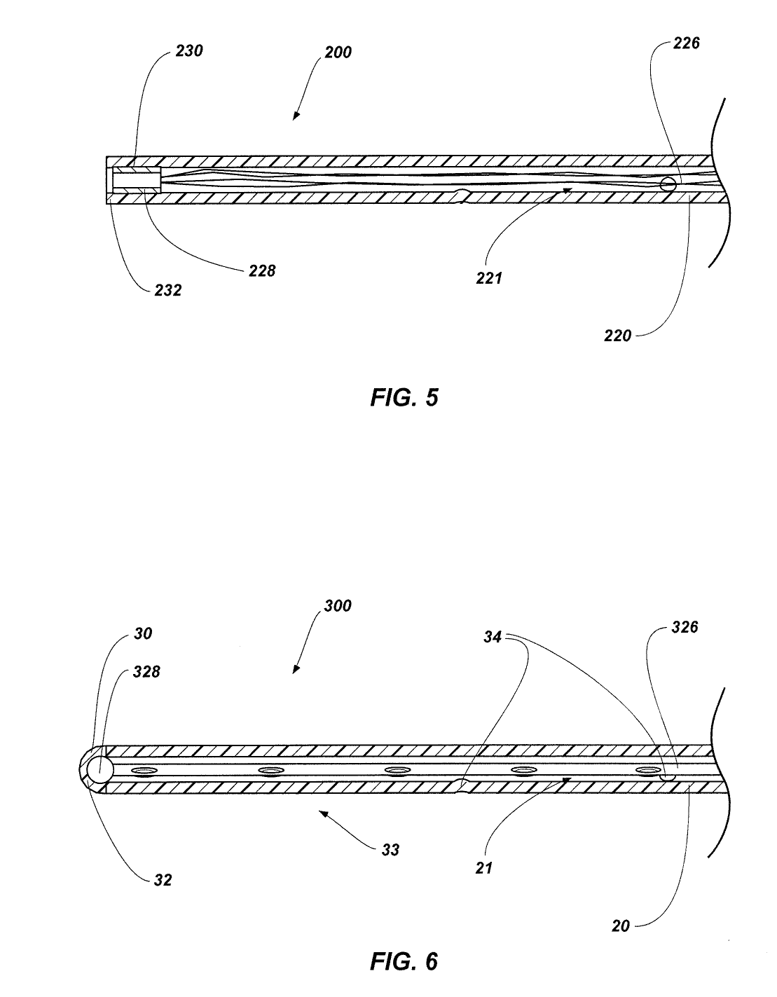

Referring again to FIG. 3, a distal end of the wires 20 (e.g., terminal end forming the end or extremity of the at least one wire 20) may include an enlarged end 28 (e.g., an end having a cross-sectional dimension, such as, for example, a diameter or thickness that is greater than a cross-sectional dimension of the wires 26). For example, the enlarged end 28 of the wires 26 may be formed as a bead (e.g., a welded bead formed substantially as a ball or spherical member). In such an embodiment, a welding process (e.g., a tungsten inert gas welding process) may be used to form the enlarged end 28 on the wires 26. In embodiments including a plurality of wires 26, the enlarged end 28 may act to couple the distal end of each individual wire 26. It is noted that while the embodiment of FIG. 3 shows the enlarged end 28 of the wires 26 formed as a bead, in other embodiments, the enlarged end may comprise any number of suitable structures (e.g., any structure that may increase the echogenic properties of the catheter 10 or secure a distal end of the wires 26 to the catheter tub 20 such as, for example, the enlarged end 228 discussed below with reference to FIG. 5). As a further example, the enlarged end 28 may comprise any of the echogenic structures described in U.S. Patent Application Publication US2011/0172542 A1, published Jul. 14, 2011, the disclosure of which is hereby incorporated herein in its entirety by this reference. In such embodiments, the enlarged end 28 may comprise one or more of a spring, a structure forming an uneven surface, a braided mesh, and a folded-over cord located within the catheter tube 20.

As further shown in FIG. 3, a portion of the wires 26 may be formed within the catheter tube 20. For example, the enlarged end 28 of the wires 26 may be formed at least partially within the catheter tube 20. In such embodiments, the wires 26 may be secured to the catheter tube 20 at or proximate the distal end 32 of the catheter 10. Such an embodiment may provide a means for removing broken portions of the catheter 10 from the subject during use. The enlarged end 28 of the wires 26 may be at least partially (e.g., entirely) formed within a solid, distal end 30 of the catheter tube 20. In other embodiments, a terminal portion of the wires 26 that does not include an enlarged end may be formed within the catheter tube 20 such as, for example, as described below with reference to FIG. 4.

The enlarged end 28 of the wires 26 may be, for example, glued, heat formed or melted to the catheter tube 20 to form the enlarged end 28 of the wires 26 at least partially within the catheter tube 20. In other embodiments, the enlarged end 28 of the wires 26 may be otherwise secured within the catheter tube 20. For example, the enlarged end 28 of the wires 26 may be secured to the catheter tube 20 by forcing the enlarged end 28 of the wires 26 into the lumen 21 of the catheter tube 20. The enlarged end 28 of the wires 26 may be sized to be larger than the lumen 21 and the catheter tube 20 may be deformed (e.g., plastically deformed, elastically deformed, or combinations thereof) as the enlarged end 28 of the wires 26 is forced within the relatively smaller lumen 21.

In some embodiments, the enlarged end 28 of the wires 26 may be formed in or otherwise secured to the sidewall of the catheter tube 20 enabling fluid to pass by the enlarged end 28 and out of the distal end 32 of the catheter tube 20 (e.g., as described below with reference to FIG. 5).

A plurality of apertures or openings 34 (e.g., openings about 0.4 millimeter (“mm”) (or about 0.015 inch) in diameter) may be formed in the sidewall of the catheter 10 at the distal portion 33 thereof so as to enable the fluid transported by the catheter 10 to pass therethrough. In embodiments where the enlarged end 28 blocks the lumen 21 at the distal end 32 of the catheter tube 20 or where, as in FIG. 3, the distal end of the catheter tube 20 is formed as a solid end 30, the plurality of openings 34 in the distal portion 33 may enable fluid to pass from the lumen 21 of the catheter tube 20 to the openings 34 formed in the sidewall of the catheter tube 20 and from the openings 34 to the lumen 21 (e.g., to delivering fluids to and/or sampling fluids from a subject's body cavity or tissue). It is noted that while the embodiment of FIG. 3 shows the catheter tube 20 as having a solid end 30, in other embodiments, the catheter may include one or more openings at the distal end thereof (see, e.g., FIG. 5). For example, openings similar to the openings 34 in the sidewall of the catheter may be formed at the distal end of the catheter (e.g., one or more openings extending around the portion of the wires foamed within the distal portion of the catheter).

In some embodiments, the wires 26 at the proximal portion 36 of the catheter tube 20 may be associated with (e.g., by adhesion, welding, molding or the like) a coiled member such as, for example, a spring contained within the proximal portion 36 of the catheter tube 20. In other embodiments, the wires 26 may be free-floating (e.g., not attached to the catheter tube 20) at the proximal portion 36 of the catheter tube 20. In some embodiments, the wires 26 may be cut to be substantially flush (or slightly recessed) with respect to the proximal end of the catheter tube 20. In such an embodiment, the wires 26 may be coupled to an additional structure such as, for example, the connector hub 24 (FIG. 2).

FIG. 4 depicts an enlarged, partial cross-sectional view of a distal portion of a catheter 100 in accordance with another embodiment of the disclosure. Catheter 100 may be somewhat similar to catheter 10 discussed above with reference to FIGS. 1 through 3. As shown in FIG. 4, a portion of one or more wires 126 may be formed within a catheter tube 120 of catheter 100. For example, a terminal portion of the wires 126 that does not include an enlarged end may be formed within the solid, distal end 130 of the catheter tube 120. In some embodiments, the portion of the wires 126 proximate the distal end 132 of the catheter 100 may be secured or formed together.

FIG. 5 depicts an enlarged, partial cross-sectional view of a distal portion of a catheter 200 in accordance with yet another embodiment of the disclosure. Catheter 200 may be somewhat similar to catheters 10, 100 discussed above with reference to FIGS. 1 through 4 and, in some embodiments, may include one or more of various components thereof As shown in FIG. 5, one or more wires 226 may be positioned within a catheter tube 220 of catheter 200 to enable fluid to pass by an enlarged end 228 of the wires 26 and out of a distal end 232 of the catheter tube 220. For example, the enlarged end 228 of the wires 226 may be formed in or otherwise secured to the sidewall of the catheter tube 20 enabling fluid to pass by the enlarged end 228 and out of the distal end 232 of the catheter tube 220. In some embodiments, and as shown in FIG. 5, the enlarged end 228 of the wires 226 may be formed to enable fluid to pass thereby. For example, the enlarged end 228 may be formed as a cylindrical member (e.g., a hollow cylindrical member such as a sleeve) or a portion of a cylindrical member that is secured to the wires 226 and the catheter tube 220 while still enabling fluid to pass through the lumen 221 to the distal end 232 of the catheter tube 220 (i.e., the enlarged end 228 does not block the distal end 232 of the catheter tube 220). Such an enlarged end 228 may be formed from a similar material as the wires 226 or a dissimilar material. In other embodiments, one or more openings (e.g., similar to openings 34 in the sidewall of the catheter 10 as shown in FIG. 3) may be formed through the catheter tube 220 at the distal end 232 of the catheter 200 enabling fluid to travel around the enlarged end 228 of the wires 226.

FIG. 6 depicts an enlarged, partial cross-sectional view of a distal portion of a catheter 300 in accordance with yet another embodiment of the disclosure. Catheter 300 may be somewhat similar to catheters 10, 100, 200 discussed above with reference to FIGS. 1 through 5 and, in some embodiments, may include one or more of various components thereof. As shown in FIG. 6, an echogenic structure of the catheter 300 may be as a ribbon (e.g., a flat ribbon having a lateral width that is greater than a thickness of the ribbon or, in other words, a rectangular lateral cross section). For example, the ribbon may be formed as a flat, dimpled ribbon 326 having one or more deformities formed therein. In other words, the dimpled ribbon 326 has been deformed along its axis (e.g., by forming circular dimples therein) such that is does not extend linearly along the length of the dimpled ribbon 326 (e.g., extends in more than one direction or dimension). In some embodiments, the dimpled ribbon 326 may be formed from the same materials as the wires as discussed herein. The dimpled ribbon 326 may include an enlarged end 328 (e.g., an end having a cross-sectional dimension that is greater than a cross-sectional dimension of the dimpled ribbon 326) disposed in (e.g., secured to) the catheter tube 20 that may be similar to the enlarged ends 28, 228 discussed above. As above, one or more of the dimpled ribbons 326 and its enlarged end 328 may enhance the ability of the catheter 300 to create an echo (i.e., return a signal in an ultrasound procedure).

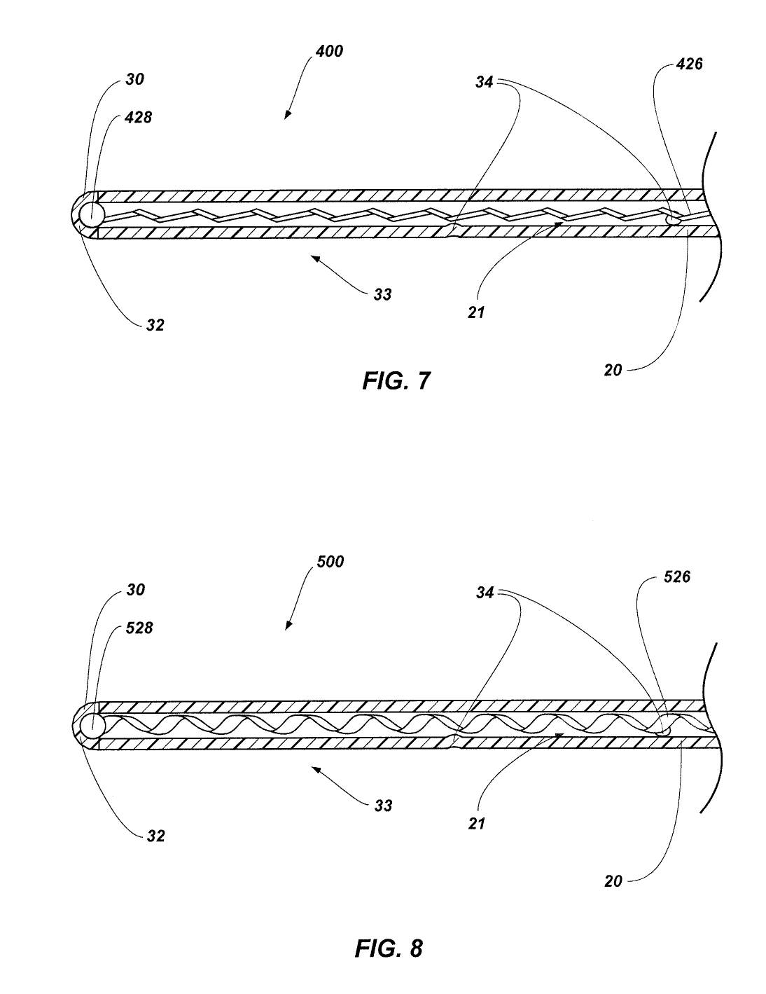

FIG. 7 depicts an enlarged, partial cross-sectional view of a distal portion of a catheter 400 in accordance with yet another embodiment of the disclosure. Catheter 400 may be somewhat similar to catheters 10, 100, 200, 300 discussed above with reference to FIGS. 1 through 6 and, in some embodiments, may include one or more of various components thereof. As shown in FIG. 7, an echogenic structure of the catheter 300 may be as a ribbon (e.g., a crimped ribbon 426). In other words, the ribbon 426 has been crimped along its length such that is does not extend linearly along the length of the ribbon 426 (e.g., extends in more than one direction or dimension). In some embodiments, the crimped ribbon 426 may be formed from the same materials as the wires as discussed herein. The crimped ribbon 426 may include an enlarged end 428 (e.g., an end having a cross-sectional dimension that is greater than a cross-sectional dimension of the ribbon 426) disposed in (e.g., secured to) the catheter tube 20 that may be similar to the enlarged ends 28, 228 discussed above. As above, one or more of the crimped ribbons 426 and its enlarged end 428 may enhance the ability of the catheter 400 to create an echo (i.e., return a signal in an ultrasound procedure).

FIG. 8 depicts an enlarged, partial cross-sectional view of a distal portion of a catheter 500 in accordance with yet another embodiment of the disclosure. Catheter 500 may be somewhat similar to catheters 10, 100, 200, 300, 400 discussed above with reference to FIGS. 1 through 7 and, in some embodiments, may include one or more of various components thereof. As shown in FIG. 8, an echogenic structure of the catheter 500 may be as a ribbon (e.g., a twisted ribbon 526). In other words, the ribbon 526 has been twisted about its axis such that is does not extend linearly along the length of the ribbon 526 (e.g., extends in more than one direction or dimension). In some embodiments, the twisted ribbon 526 may be formed from the same materials as the wires as discussed herein. The twisted ribbon 526 may include an enlarged end 528 (e.g., an end having a cross-sectional dimension that is greater than a cross-sectional dimension of the ribbon 526) disposed in (e.g., secured to) the catheter tube 20 that may be similar to the enlarged ends 28, 228 discussed above. As above, one or more of the twisted ribbons 526 and its enlarged end 528 may enhance the ability of the catheter 500 to create an echo (i.e., return a signal in an ultrasound procedure).

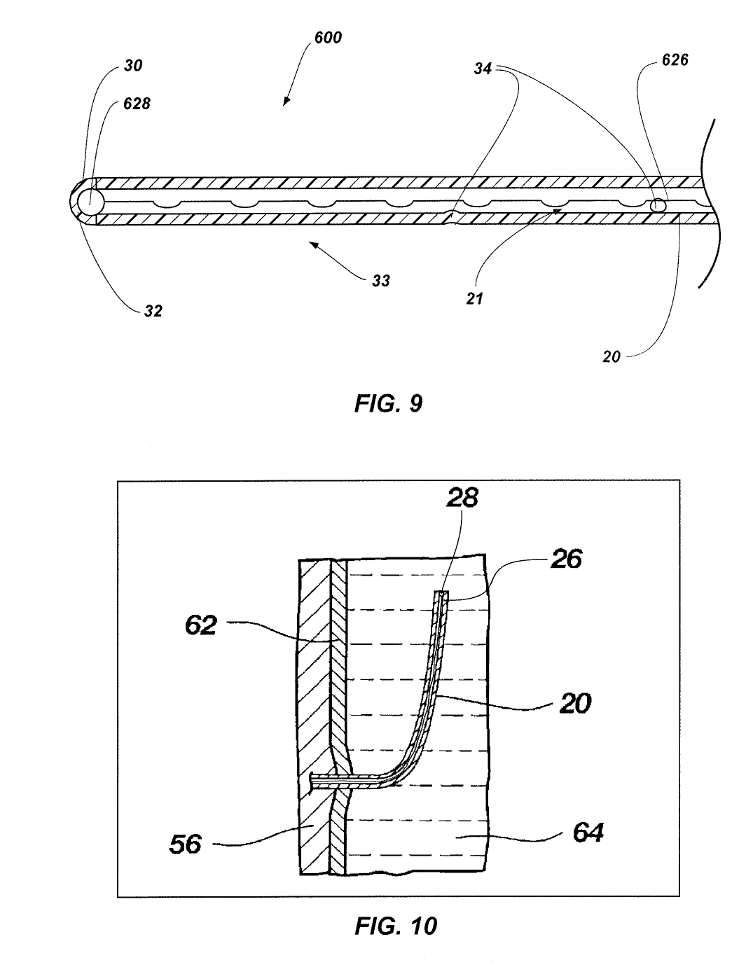

FIG. 9 depicts an enlarged, partial cross-sectional view of a distal portion of a catheter 600 in accordance with yet another embodiment of the disclosure. Catheter 600 may be somewhat similar to catheters 10, 100, 200, 300, 400, 500 discussed above with reference to FIGS. 1 through 8 and, in some embodiments, may include one or more of various components thereof. As shown in FIG. 9, an echogenic structure of the catheter 600 may be as one or more wires having one or more deformed portions (e.g., a pressed wire 626). In other words, portions of the pressed wire 626 have been plastically deformed such that portions of the pressed wire 626 are not linear (e.g., extend in more than one direction or dimension). In some embodiments, the pressed wire 626 may be formed from the same materials as the wires as discussed herein. The pressed wire 626 may include an enlarged end 628 (e.g., an end having a cross-sectional dimension that is greater than a cross-sectional dimension of the wire 626) disposed in (e.g., secured to) the catheter tube 20 that may be similar to the enlarged ends 28, 228 discussed above. As above, one or more of the pressed wires 626 and its enlarged end 628 may enhance the ability of the catheter 600 to create an echo (i.e., return a signal in an ultrasound procedure).

In some embodiments, the above-described catheters may be utilized with a clinical procedural tool such as a flexible spinal needle assembly. Such flexible spinal needle assemblies are disclosed in, for example, United States Patent Application Publication US 2008/0065017 A1, which application was filed Oct. 31, 2007 and entitled “Method of Using Flexible Spinal Needle Assemblies,” the disclosure of which is hereby incorporated herein in its entirety by this reference. For example, as described in the incorporated US 2008/0065017 A1, a flexible spinal needle assembly may include a catheter such as a flexible needle. The flexible needle may include an echogenic structure such as those described herein within the flexible needle. In some embodiments, the flexible needle may include a cord (similar to the cords 26, 126 described herein) comprised of braided wires (e.g., 16 count 0.0254 millimeter (0.001 inch) braided wires) forming an echogenic structure at a distal portion of the flexible needle.

In use, embodiments of the present disclosure may increase echogenicity of the catheter for improved imaging by an ultrasound sensor used to guide placement of the catheter with the wires, the enlarged end of the wires, or the combination of the wires and the enlarged end of the wires. For example, referring to FIG. 10, a catheter tube 20 is shown in use in proximate neural structures 64 of the nervous system (e.g., a neural structure of the peripheral nervous systems) of a subject (e.g., a mammal, such as a human). The catheter 10 may be placed within a subject through skin 56 and other outer layers 62 (e.g., muscle, fat, etc.) using, for example, an introducer needle (e.g., a flexible introducer cannula (FIC)), a flexible needle as discussed above, or any other suitable types of needles. Under ultrasound guidance (sensor not shown), the catheter tube 20 may be advanced into the subject to be positioned proximate (e.g., adjacent) one or more neural structures 64. The proximal portion of the catheter tube 20 is maintained outside the subject's body and may be coupled to any desired tubing, syringe, etc. If any portion of the catheter tube 20 in the subject is broken, it may be retrieved by pulling on the wires 26. In addition, if the catheter tube 20 should become kinked, the wires 26 may allow the flow of fluids through the catheter.

As also can been seen in FIG. 10, an echogenic structure (e.g., the wires 26, 126, 226, 626, the ribbons 326, 426, 526, the enlarged ends 28, 228, 328, 428, 528, 628, or combinations thereof as described above with references to FIGS. 3 and 5 through 10) located in the catheter tube 20 may enhance the ability of portions of the catheter tube 20 (e.g., the distal portion) to create an echo (i.e., to be echogenic and return a signal in an ultrasound procedure). By increasing the ability of the catheter tube 20 to create an echo, the distal portion of the catheter tube 20 may be relatively more easily identified and located during a procedure utilizing ultrasonic guidance to place a catheter within a subject.

Methods of forming catheters having improved echogenicity may include the exemplary methods and materials such as those discussed above and the following. For example, the catheter tube may be formed from a flexible pre-tapered, pre-holed TECOTHANE® 55D polyurethane tubing (e.g., 0.9 mm (0.035 inch) outer diameter, 0.6 mm (0.025 inch) inner diameter), nylon (e.g., PEBAX® 55D), a polyimide (e.g., a polyimide having a durometer of 72D), etc. In other embodiments, it can be made of a synthetic absorbable polymer in a manner similar to that disclosed in U.S. Pat. No. 5,129,889 to Hahn et al. (Jul. 14, 1992), the disclosure of which is hereby incorporated herein in its entirety by this reference. The cord may be, for example, twisted 0.009 inch 304 stainless steel or 0.25 mm (0.010 inch) diameter nylon. The enlarged portion may have a diameter of about 0.86 mm (0.035 inches).

1. A catheter for utilization with a portion of a nervous system of a subject, the catheter comprising:
a catheter tube comprising a proximal end, a distal end, and a lumen formed therebetween; and
a structure disposed within the lumen of the catheter tube and extending from a proximal portion of the catheter tube to a distal portion of the catheter tube, the structure comprising an enlarged distal end having a cross-sectional dimension that is greater than a cross-sectional dimension of the structure, the enlarged distal end being at least partially formed in the catheter tube proximate the distal end of the catheter tube, wherein the structure comprises at least two substantially discrete sets of wires.

2. The catheter of claim 1, wherein the enlarged distal end of the structure comprises a bead formed on the distal end of the structure.

3. The catheter of claim 2, wherein the distal end of the catheter tube comprises a solid, distal end, and wherein the catheter tube comprises a plurality of apertures formed in a sidewall of the catheter tube at the distal portion of the catheter tube enabling fluid transported by the catheter to pass therethrough.

4. The catheter of claim 3, wherein a majority of the bead formed on the distal end of the structure is embedded in the solid distal end of the catheter tube.

5.-7. (canceled)

8. The catheter of claim 1, wherein a distal end of the at least two sets of wires comprises a weld bead, wherein each wire of the at least two sets of wires terminates at the weld bead, and wherein a remaining portion of each set of the at least two sets of wires extends separately through the catheter tube.

9. A catheter for use with a portion of a
nervous system of a subject, the catheter comprising:
a catheter tube comprising a proximal end, a distal end, and a lumen formed therebetween; and
a structure disposed within the lumen of the catheter tube and extending from a proximal portion of the catheter tube to a distal portion of the catheter tube, the structure comprising an enlarged distal end having a cross-sectional dimension that is greater than a cross-sectional dimension of the structure, the enlarged distal end being at least partially formed in the catheter tube proximate the distal end of the catheter tube;
wherein the structure comprises at least one of at least one deformed wire that extends along the length of the deformed wire in at least two dimensions and a deformed ribbon that extends along the length of the deformed ribbon in at least two dimensions.

10. The catheter of claim 9, wherein the deformed ribbon comprises at least one of a dimpled ribbon, a crimped ribbon, and a twisted ribbon.

11. A catheter having improved echogenicity for use with a portion of a nervous system of a subject, the catheter comprising:
a catheter tube having an open proximal end in communication with a lumen and a distal end, wherein the distal end of the catheter tube comprises an opening; and
an echogenic structure extending through the lumen from the proximal end to proximate the distal end of the catheter tube, the echogenic structure terminating in a weld bead at least partially embedded within a distal portion of the catheter tube proximate the distal end of the catheter tube, wherein the echogenic structure and the weld bead provide improved imaging by an ultrasound sensor used to guide placement of the catheter and means for removing broken portions of the catheter during use.

12. (canceled)

13. (canceled)

14. A catheter configured for at least one of delivering fluid to and sampling fluid from a portion of a nervous system of a subject, the catheter comprising:
a catheter tube comprising a proximal end, a distal end, and a lumen formed therebetween, the catheter tube having a plurality of apertures formed in a sidewall of the catheter tube at a distal portion of the catheter tube enabling fluid transported by the catheter to pass therethrough; and
at least two discrete sets of wires disposed within the lumen of the catheter tube, each set comprising a twisted pair of wires extending from a proximal portion of the catheter tube to a distal portion of the catheter tube, wherein the at least two discrete sets of wires comprise an enlarged terminal end having a diameter that is greater than a cross-sectional diameter of at least one wire of the at least two discrete sets of wires, the enlarged terminal end being at least partially formed in the catheter tube proximate the distal end of the catheter tube, and wherein a remaining portion of each set of the at least two discrete sets of wires extends through the catheter tube separate from at least another set of the at least two discrete sets of wires.

15. A method of forming a tool for a clinical procedure performed upon a portion of a peripheral nervous system of a subject utilizing ultrasound guidance, the method comprising:
forming a catheter having a lumen extending from a proximal end of the catheter to a solid distal end of the catheter;
disposing an echogenic structure within the lumen of the catheter extending from a proximal portion of the catheter to a distal portion of the catheter, wherein the echogenic structure comprises two twisted pairs of wires; and
forming an enlarged end of the echogenic structure within the catheter proximate the solid distal end of the catheter.

16. The method according to claim 15, wherein the echogenic structure further comprises a deformed ribbon.

17. (canceled)

18. The method according to claim 15, further comprising terminating each wire of the at least two twisted pairs of wires with the enlarged end.

19. (canceled)

20. (canceled)

21. A method of ultrasound guidance for a clinical procedure performed upon a portion of a peripheral nervous system of a subject utilizing the catheter of claim 14, the method comprising:
inserting the catheter into the subject; and
guiding placement of the catheter within the subject utilizing an ultrasound sensor and the echogenicity of at least a portion of the structure within the lumen of the catheter.

22. A method of ultrasound guidance for a clinical procedure performed upon a portion of a peripheral nervous system of a subject utilizing the catheter of claim 1, the method comprising:
inserting the catheter into the subject; and
guiding placement of the catheter within the subject utilizing an ultrasound sensor and the echogenicity of at least a portion of the structure within the lumen of the catheter.

23. A method of ultrasound guidance for a clinical procedure performed upon a portion of a peripheral nervous system of a subject utilizing the catheter of claim 9, the method comprising:
inserting the catheter into the subject; and
guiding placement of the catheter within the subject utilizing an ultrasound sensor and the echogenicity of at least a portion of the structure within the lumen of the catheter.

24. A method of ultrasound guidance for a clinical procedure performed upon a portion of a peripheral nervous system of a subject utilizing the catheter of claim 11, the method comprising:
inserting the catheter into the subject; and
guiding placement of the catheter within the subject utilizing an ultrasound sensor and the echogenicity of at least a portion of the structure within the lumen of the catheter.
